SEQUENCE LISTING (5 sequences)

SEQ ID NO: 1 - DNA, 2930 bp, Homo sapiens
  CDS (930)...(2546)
aatcgggctgccttatcgccaagctccttcaggagaacaaagaacaggccattaccctggagaagactggcaactgattttacccacaagcccaaacctcagggatttcagtatctactagtctgggtagatactttcacgggttgggcagaggccttcccctgtaggacagaaaaggcccaagaggtaataaaggcactagttcatgaaataattcccagattcggacttccccgaggcttacagagtgacaatagccctgctttccaggccacagtaacccagggagtatcccaggcgttaggtatacgatatcacttacactgcgcctgaaggccacagtcctcagggaaggtcgagaaaatgaatgaaacactcaaaggacatctaaaaaagcaaacccaggaaacccacctcacatggcctgctctgttgcctatagccttaaaaagaatctgcaactttccccaaaaagcaggacttagcccatacgaaatgctgtatggaaggcccttcataaccaatgaccttgtgcttgacccaagacagccaacttagttgcagacatcacctccttagccaaatatcaacaagttcttaaaacattacaaggaacctatccctgagaagagggaaaagaactattccacccttgtgacatggtattagtcaagtcccttccctctaattccccatccctagatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacaattatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagagcggtcgtcggccaacctccccaacagcacttaggttttcctgttgagatgggggactgagagacaggactagctggatttcctaggctgactaagaatccctaagcctagctgggaaggtgaccacatccacctttaaacacggggcttgcaacttagctcacacctgaccaatcagagagctcactaaaatgctaattaggcaaaaacaggaggtaaagaaatagccaatcatctattgcctgagagcacagcaggagggacaatgatcgggatataaacccaagtcttcgagccggcaacggcaaccccctttgggtcccctccctttgtatgggagctctgttttcatgctatttcactctattaaatcttgcaactgc

SEQ ID NO: 2 - protein, 538 aa, Homo sapiens [sequence omitted]

SEQ ID NO: 3 - DNA, 20 bp, Homo sapiens
cacaaccaactcccaatgca

SEQ ID NO: 4 - DNA, 23 bp, Homo sapiens
agagccattcaaacaacgatagg

SEQ ID NO: 5 - DNA, 30 bp, Homo sapiens
ctcctcccacacaaatagtctgcctaccct

(19) United States
(12) Patent Application Publication
(10) Pub. No.: US 2002/0102530 A1
(43) Pub. Date: Aug. 1, 2002

(54) Methods and compositions for diagnosing and treating preeclampsia and gestational trophoblast disorders

(76) Inventors: James C. Keith JR., Andover, MA (US); John M. McCoy, Reading, MA (US); Sha Mi, Belmont, MA (US)

Correspondence Address: LAHIVE & COCKFIELD, 28 STATE STREET, BOSTON, MA, 02109, US

(21) Appl. No.: 09/902,535

(22) Filed: Jul. 9, 2001

Related U.S. Application Data
(60) Provisional application No. 60/216,657, filed on Jul. 7, 2000.

(51) Int. Cl. C12Q 1/00; C12Q 1/68; G01N 33/567

(52) U.S. Cl. 435/4; 435/6; 435/7.21

(57) ABSTRACT

The invention provides methods for identifying compounds which are modulators of syncytin expression. The invention also provides methods for diagnosing a subject who is suffering from or at risk for developing preeclampsia, and kits for diagnosing such a subject. The invention further provides methods for treating a subject who is suffering from or at risk for developing preeclampsia. The invention still further provides methods for treating a subject who is suffering from a gestational trophoblast disorder.

### RELATED APPLICATIONS

This application claims the benefit of U.S. Provisional Application Serial No. 60/216,657, filed Jul. 7, 2000, the entire contents of which are incorporated herein by this reference. This application is also related to U.S. patent application Ser. No. 09/175,928, filed Oct. 20, 1998 (pending), the entire contents of which are incorporate herein by this reference.

### BACKGROUND OF THE INVENTION

Development of placentation and successful pregnancy depend on coordinated interactions between the uterine epithelium, the maternal decidua and myometrium, and the invasive properties of the fetal trophoblast. Preeclampsia is a hypertensive, multi-system disorder of pregnant women that affects approximately 6% of first pregnancies and 1-2% of all pregnancies (MacGillivary, I., Preeclampsia: the hypertensive disease of pregnancy. W. B. Saunders, Philadelphia 1987:17). Preeclampsia is a major cause of maternal and fetal mortality and morbidity, and is a disease unique to human beings during pregnancy. Hospitalization, strict bed rest, magnesium sulfate administration to prevent convulsions, and prompt delivery remain as the current standard of therapy for preeclampsia. Despite many years of study, the causes of preeclampsia are unclear.

The hallmarks of preeclampsia include hypertension, proteinuria, and edema. Underlying these clinical manifestations, placental maladaptation and body-wide endothelial cell dysfunction occur (Khong, T. Y. et al. (1986) Br. J. Obstet. Gynecol. 93:1049-1059; Roberts, J. M. et al. (1991) Am. J. Hypertens. 4:700-708). Failure of trophoblastic invasion into myometrial segments of maternal spiral arteries and the production of cytotoxic mediators which cause systemic endothelial damage also seem to be implicated.

Trophoblasts are a unique cell type in that they share characteristics of both normal and neoplastic tissue. During normal development, like neoplastic cells, human trophoblasts invade through the extracellular matrix into the myometrial portion of spiral arteries. However, unlike neoplastic cells, which endlessly invade and finally spread to other organs, the invasive properties of the trophoblasts are eventually brought under control, further cell differentiation proceeds, and cell senescence occurs. In normal gestation, trophoblasts convert spiral arteries into uteroplacental arteries by the above process (Pijnenborg et al., in Trophoblast Research (Denker and Aplin, eds.) Plenum Press, New York, p. 333 (1990)). Uteroplacental arteries then dilate approximately 30-fold as large as the spiral arteries. Resulting hemodynamic changes enable the placental bed to satisfy the increased demand for oxygen from the fetus during the latter stages of gestation. In preeclamptic women, however, spiral arteries are not properly converted into uteroplacental arteries due to the failure of the second wave of trophoblastic migration into the myometrium at the beginning of the second trimester (Khong et al. (1986) Br. J. Obstet. Gynecol. 93:1049-1059). As a result, preeclamptic women typically demonstrate a high-resistance, high-pressure, and low-flow state with intact, non-dilated spiral arteries (Robertson et al. (1986) Am. J. Obstet. Gynecol. 155:401-412), and demonstrate a wide variety of clinical syndromes. Thus, abnormal behavior of the fetus-derived trophoblast appears to be a central aspect of the disease.

### SUMMARY OF THE INVENTION

The present invention relates to methods and compositions for diagnosing and treating preeclampsia and gestational trophoblast disorders using modulators of syncytin expression. Syncytin is a human gene derived from the envelope gene of a recently identified human endogenous defective retrovirus, HERV-W (Blond, J. L. et al. (1999) J. Virol. 73:1175-85). Syncytin is only expressed in placental tissue, and the major site of syncytin expression is the placental syncytiotrophoblast (Mi, S. et al. (2000) Nature 403:785-789; PCT International Publication No. WO 99/60020), which is a fused multinuclear syncytium originating from cytotrophoblast cells. Previous data suggest that syncytin may mediate placental cytotrophoblast fusion in vivo and may play an important role in human placental morphogenesis (Mi, S. et al. (2000) supra; PCT International Publication No. WO 99/60020). The present invention is based, at least in part, on the discovery that syncytin expression is dramatically reduced in preeclampsia, and it is also mis-localized to the apical syncytiotrophoblast membrane.

In one embodiment, the present invention provides methods for identifying compounds which are capable of treating or preventing preeclampsia that include contacting a cell (e.g., a placental cell; a cytotrophoblast; a syncytiotrophoblast; a cell comprising or transfected with a nucleic acid molecule encoding a syncytin polypeptide; or a cell comprising or transfected with a nucleic acid molecule comprising at least one syncytin regulatory element operatively linked to a reporter gene) and determining whether syncytin expression or activity in the cell is modulated, for example, as compared to a suitable control (e.g., untreated cells or cells treated with a control). In a preferred embodiment, syncytin expression or activity is increased.

A further embodiment of the invention provides compounds identified according to the above-described methods.

Another embodiment of the invention provides methods for diagnosing a subject who is suffering from or at risk for developing preeclampsia, including contacting a biological sample (e.g., a serum sample, an amniotic fluid sample, a chorionic villus sample, a placental sample, a tissue sample, a biological fluid sample, a tumor sample, an endothelial cell sample, a plasma sample, or a blood sample), or isolate thereof, with an agent capable of detecting syncytin mRNA or polypeptide, such that the presence of syncytin mRNA or polypeptide is detected in the biological sample, or isolate thereof, thereby determining whether the subject is suffering from or at risk for developing preeclampsia. In a preferred embodiment, syncytin polypeptide is detected in the biological sample. In another preferred embodiment, the biological sample is an amniotic fluid sample, a chorionic villus sample, or a placental sample. In a more preferred embodiment, the biological sample is a serum sample.

In a further embodiment, the invention provides a method for determining the level of syncytin mRNA or polypeptide in a biological sample. In still another embodiment, the invention provides a method for comparing the level of syncytin mRNA or polypeptide in a biological sample with the level of syncytin mRNA or polypeptide from a suitable or appropriate control.

In still a further embodiment, the invention provides methods for diagnosing a subject who is suffering from or is at risk for developing preeclampsia by further determining the levels of M-CSF and/or TNFα in a biological sample from the subject, or an isolate thereof, and comparing them to a suitable or appropriate control.

In another embodiment, the invention provides kits for diagnosing a subject suffering from or at risk for developing preeclampsia including an agent for detecting and/or determining the level of syncytin mRNA or polypeptide in a biological sample, or isolate thereof, and comparing the level of syncytin to a suitable or appropriate control. Such kits can further include instructions for use.

In another embodiment, the invention provides methods for treating a subject suffering from or at risk for developing preeclampsia with a therapeutically effective amount of a syncytin modulator. In a further embodiment, the invention provides methods for treating a subject suffering from or at risk for developing preeclampsia with therapeutically effective amounts of a syncytin modulator and, optionally, M-CSF and/or a TNFα inhibitor (e.g., etanercept).

In another embodiment, the invention provides methods for treating a subject suffering from a gestational trophoblast disorder (e.g., a missed or incomplete abortion, choriocarcinoma, hydatiform mole, or placental site tumor).

Other features and advantages of the invention will be apparent from the following detailed description and claims.

### DETAILED DESCRIPTION OF THE INVENTION

The present invention is based, at least in part, on the discovery that the expression of the syncytin gene in preeclampsia is dramatically reduced. The invention is also based of the discovery that the syncytin protein is aberrantly localized to the apical syncytiotrophoblast villous membrane in preeclamptic placental tissue, as opposed to its normal location on the basal membrane.

As used herein, “syncytin” refers to a human gene derived from the envelope gene of a recently identified human endogenous defective retrovirus, HERV-W (Blond, J. L. et al. (1999) J. Virol. 73:1175-85). Syncytin is only expressed in placental tissue, and the major site of syncytin expression is the placental syncytiotrophoblast (Mi, S. et al. (2000) Nature 403:785-789; PCT International Publication No. WO 99/60020), which is a fused multinuclear syncytium originating from cytotrophoblast cells. Previous data suggest that syncytin may mediate placental cytotrophoblast fusion in vivo and may play an important role in human placental morphogenesis (Mi, S. et al. (2000) supra; PCT International Publication No. WO 99/60020).

As used herein, the term “preeclampsia” includes a hypertensive, multi-system disorder of pregnant women, characterized by hypertension, proteinuria, edema, body-wide endothelial cell dysfunction, and placental maladaptation. Morphologic abnormalities of the placental villi are frequently described in placentas derived from preeclamptic pregnancies (Fox H., Pathology of the Placenta. W B Saunders, London 1997:151-159). Such abnormalities include disturbances in the syncytiotrophoblast such as increased numbers of syncytial knots (abnormal accumulations of syncytiotrophoblast nuclei) and the progressive proliferation of the villous cytotrophoblast.

As used interchangeably herein, the terms “trophoblast” and “cytotrophoblast” include cells of fetal origin which differentiate to form various tissues of the placenta, including the syncytiotrophoblast (as defined herein) on the villous surface of the placenta. In normal mammals, for example, a pregnant female mammal, the villous trophoblast cells are located immediately below the syncytiotrophoblast, and they proliferate and then fuse with the growing syncytiotrophoblast throughout progression of the pregnancy, thus allowing the syncytiotrophoblast to enlarge to accommodate placental villous elongation and branching. Trophoblasts, particularly those in the extravillous region of the placenta, also include cells that differentiate into intermediate trophoblast cells and then into multinucleated intermediate trophoblastic cells (Shih, I. M. and Kurman, R. J. (1997) Verh. Dtsch. Ges. Pathol. 81:266-72).

As used herein, the term “syncytiotrophoblast” includes a multinucleate, fused cell type of fetal origin. Syncytiotrophoblasts arise by cell fusion from cytotrophoblasts and constitute the boundary layer between maternal and fetal tissue. They are important in maternal-fetal exchange, in tissue remodeling during placental development, and in protecting the developing fetus from the maternal immune response (Cross, J. C. et al. (1996) Science 266:1508-1518; Lala, P. K. and Hamilton, G. S. (1996) Placenta 17:545-555; Munn, D. H. et al. (1998) Science 281:1191-1193).

The present invention is based, at least in part, on the discovery that syncytin expression in preeclampsia is dramatically reduced, as measured by in situ hybridization, Northern blot, and quantitative RT-PCR analysis. The invention is also based of the discovery that the syncytin protein is localized improperly to the apical syncytiotrophoblast villous membrane in preeclamptic placental tissue, as opposed to its normal location on the basal membrane.

Accordingly, the present invention features methods for identifying compounds which modulate the expression or activity of syncytin. As used interchangeably herein, the terms “expression of syncytin” and “syncytin expression” refer to the syncytin expressed or present in a biological sample, isolate, tissue, cell, or biological fluid. The syncytin may be in the form of nucleic acid molecules (e.g., mRNA) or polypeptide molecules. Use used interchangeably herein, the terms “activity of syncytin” and “syncytin activity” include one or more of the following activities: the ability to mediate the fusion of one cell with another cell (e.g., two of the same cells or two different cells, for example, a placental cell, a trophoblast, a syncytiotrophoblast, a cell comprising or transfected with a nucleic acid molecule encoding a syncytin gene, or a cell comprising or transfected with a nucleic acid molecule comprising at least one syncytin regulatory element operatively linked to a reporter gene) and the ability to localize to the basal membrane of a syncytiotrophoblast.

In another embodiment, the invention features prognostic and diagnostic methods useful in the prediction, detection, and treatment of preeclampsia and gestational trophoblast disorders. The invention further features kits useful in the prediction and detection of preeclampsia. As defined herein, the term “gestational trophoblast disorder” includes pathologies, disorders, and conditions involving or affecting the placenta or trophoblast cells. Such conditions include, but are not limited to, preeclampsia, gestational hypertension, choriocarcinoma, missed or incomplete abortion, placental site tumors, and hydatiform mole.

In one embodiment, the invention features a prognostic method for identifying a subject at risk for preeclampsia involving obtaining a biological sample from the subject and determining the level of syncytin in the biological sample. As used herein, a “biological sample” refers to a sample of biological material obtained from a subject, preferably a human subject, or present within a subject, preferably a human subject, including a tissue, tissue sample, or cell sample (e.g., a chorionic villus sample or a tissue biopsy, for example, an aspiration biopsy, a brush biopsy, a surface biopsy, a needle biopsy, a punch biopsy, an excision biopsy, an open biopsy, an incision biopsy, or an endoscopic biopsy), tumor, tumor sample, or biological fluid (e.g., blood, serum, plasma, amniotic fluid, urine, lymph, or spinal fluid).

As used herein, a “tissue sample” refers to a portion, piece, part, segment, or fraction of a tissue which is obtained or removed from an intact tissue of a subject, preferably a human subject. For example, tissue samples can be obtained from the skin, endothelium, vasculature, chorionic villus, placenta, uterus, vagina, or connective tissue. As used herein, a “tumor sample” refers to a portion, piece, part, segment, or fraction of a tumor, for example, a tumor which is obtained or removed from a subject (e.g., removed or extracted from the tissue of a subject), preferably a human subject. A tumor sample can be obtained, for example, from a choriocarcinoma.

The present invention also encompasses the use of isolates of a biological sample in the methods of the invention. As used herein, an “isolate” of a biological sample (e.g., an isolate of a serum sample or a tissue or tumor sample) refers to a material or composition (e.g., a biological material or composition) which has been separated, derived, divided, extracted, purified, or isolated from the sample and preferably is substantially free of undesirable compositions and/or impurities or contaminants associated with the biological sample. Preferred isolates include, but are not limited to, DNA (e.g., cDNA or genomic DNA), RNA (e.g., mRNA), and protein (e.g., purified protein, protein extracts, or polypeptides). Additional preferred isolates include cells, as well as biological fluids (e.g., blood, serum, amniotic fluid, urine, plasma, lymph, or spinal fluid).

The present invention features agents which are capable of detecting syncytin mRNA or polypeptide such that the presence of syncytin mRNA or polypeptide is detected. As defined herein, an “agent” refers to a substance which is capable of identifying or detecting syncytin in a biological sample (e.g., identifies or detects syncytin mRNA, DNA, polypeptide, or activity). In one embodiment, the agent is a labeled or labelable antibody which specifically binds to a syncytin polypeptide. As used herein, “labeled or labelable” refers to the attaching or including of a label (e.g., a marker or indicator). Markers or indicators include, but are not limited to, for example, radioactive molecules, fluorescent molecules, calorimetric molecules, and enzymatic molecules which produce detectable changes in a substrate. In one embodiment, the agent is an antibody which specifically binds to all or a portion of a syncytin polypeptide. As used herein, the phrase “specifically binds” refers to binding of, for example, an antibody to an epitope, antigen, or antigenic determinant in such a manner that binding can be displaced or competed with a second preparation of identical or similar epitope, antigen, or antigenic determinant. In an exemplary embodiment, the agent is a monoclonal antibody which specifically binds to all or a portion of a syncytin polypeptide. In another embodiment, the antibody is a polyclonal antibody.

In yet another embodiment, the agent is a labeled or labelable nucleic acid probe capable of hybridizing to syncytin mRNA. For example, the agent can be an oligonucleotide primer for the polymerase chain reaction which flanks or lies within the nucleotide sequence encoding the human syncytin. In a preferred embodiment, the biological sample being tested is an isolate, for example, RNA. In another embodiment, the isolate (e.g., RNA) is subjected to an amplification process which results in amplification of the syncytin nucleic acid. As defined herein, an “amplification process” is designed to strengthen, increase, or augment a molecule within an isolate. For example, where the isolate is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Another aspect of the invention features a prognostic or diagnostic method for determining whether a subject is at risk for developing preeclampsia which involves contacting the biological sample obtained from the subject (or an isolate of the sample) with an agent capable of detecting syncytin mRNA or polypeptide such that the presence of syncytin mRNA or polypeptide is detected in the biological sample or isolate, thereby determining whether the subject is at risk for developing preeclampsia. As used herein, a subject “at risk for developing preeclampsia” includes a subject who has a higher probability of developing preeclampsia when compared to an average representative of the population. A subject's “risk of developing” preeclampsia can be based on an analysis of empirical criteria, or alternatively, can be based on a determination of altered syncytin expression or activity, for example, in a biological sample from said subject.

In another embodiment, the prognostic or diagnostic methods of the present invention further involve determining the level of syncytin mRNA or polypeptide in the biological sample or isolate. As used herein, “determining the level” includes measuring an amount (e.g., making a quantitative determination) or making a qualitative determination (e.g., a determination of the presence versus the absence of syncytin mRNA or polypeptide). In yet another embodiment, the prognostic or diagnostic methods of the present invention involve comparing the level of syncytin mRNA or polypeptide in the sample or isolate with the level of syncytin mRNA or polypeptide in an appropriate control sample. As used herein, the phrase “comparing the level” includes evaluating, balancing, or contrasting the amount or presence of, for example, syncytin mRNA or polypeptide in a first sample (e.g., a test sample) with the amount or presence of syncytin mRNA or polypeptide in a second sample (e.g., a control sample). In a preferred embodiment, the amount of syncytin mRNA or polypeptide in a test sample is compared with averaged syncytin mRNA or polypeptide levels from samples taken from pregnant subjects who have had normal pregnancies. In a further embodiment, the methods of the present invention comprise determining the levels of syncytin mRNA or polypeptide, along with determining the levels of M-CSF and/or TNFα mRNA or polypeptide, and comparing the level of each factor with its respective level in pregnant subjects who later went on to have normal pregnancies.

In another embodiment, the present invention features methods of treating a subject using a syncytin modulator. In one embodiment, the subject being treated has been diagnosed as having or being at risk for preeclampsia using one of the methods of the present invention. In another embodiment, the subject being treated has a gestational trophoblast disorder (e.g., choriocarcinoma, hydatiform mole, placental site tumor, or missed or incomplete abortion).

In one embodiment, the subject is treated with a therapeutically effective amount of a molecule identified as a syncytin modulator. In a preferred embodiment, the syncytin modulator increases expression of syncytin in the subject. In another embodiment, the subject is also treated with a therapeutically effective amount of M-CSF and/or a TNFα inhibitor. In a preferred embodiment, the TNFα inhibitor is etanercept. As used herein, a “TNFα inhibitor” is defined as a compound or agent that inhibits the biological activity of TNFα. As used herein, the “biological activity of TNFα” is defined as the ability of TNFα to (1) bind to its natural receptor(s) on a cell and/or (2) activate signaling in a cell through its receptor(s). As used herein, a “therapeutically effective amount” of a compound is the amount of a compound that is effective in treating a condition that is treatable by administration of the compound. In a preferred embodiment, the condition is preeclampsia. In another embodiment, the condition is a gestational trophoblast disorder (e.g., choriocarcinoma, hydatiform mole, placental site tumor, or missed or incomplete abortion). In one embodiment, a therapeutically effective amount of a compound is the amount of the compound sufficient to bring serum syncytin, M-CSF, and/or TNFα levels to normal levels, as compared to a suitable control (e.g., the levels detected in a normal pregnant subject or average levels from normal pregnant subjects).

In yet another embodiment, the invention features kits for detecting the presence of syncytin in a biological sample (or isolate of a sample) including an agent (e.g., a labeled or labelable agent) capable of detecting syncytin mRNA or polypeptide in a biological sample. In one embodiment, the kit further includes a means for determining the amount of syncytin in the sample. In another embodiment, the agent of the kit is an antibody capable of specifically binding to syncytin polypeptide. In another embodiment, the agent of the kit is a nucleic acid probe capable of hybridizing to syncytin mRNA. In yet another embodiment, the kit further includes a suitable or appropriate standard or control. In yet a further embodiment, the kit includes instructions for use.

Various aspects of the invention are described in further detail in the following subsections:

### I. Methods for Identifying Syncytin Modulators

The invention provides methods (also referred to herein as a “screening assays”) for identifying modulators, e.g., candidate or test compounds or agents (e.g., peptides, peptidomimetics, small molecules, or other drugs) which modulate the expression or activity of syncytin.

In one embodiment, the invention provides assays for screening candidate or test compounds which modulate expression of syncytin mRNA or polypeptide. In another embodiment, the invention provides assays for screening candidate or test compounds which modulate the activity of syncytin polypeptide. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer, or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which is capable of expressing syncytin is contacted with a test compound, and the ability of the test compound to modulate syncytin expression or activity is determined. The cell can be any type of cell that can express syncytin or respond in a syncytin-dependent manner. For example, the cell can be a placental cell such as a trophoblast cell or a syncytiotrophoblast cell. The cell may also be a placental or non-placental cell comprising or transfected with a nucleic acid molecule encoding a syncytin polypeptide or a nucleic acid molecule comprising at least one syncytin regulatory element operatively linked to a reporter gene (e.g., a nucleic acid molecule encoding a detectable marker, such as LacZ or luciferase). The nucleic acids transfected into the cells of the screening assays described herein are preferably contained in a vector, preferably an expression vector.

Therefore, another aspect of the invention pertains to vectors, for example recombinant expression vectors, containing a syncytin nucleic acid molecule or vectors containing a nucleic acid molecule which encodes a syncytin polypeptide (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. Reporter genes

In one embodiment, modulators of syncytin expression are identified in a method wherein a cell is contacted with a candidate compound and the expression of syncytin mRNA or protein in the cell is determined. The level of expression of syncytin mRNA or protein in the presence of the candidate compound can be compared to the level of expression of syncytin mRNA or protein in the absence of the candidate compound.

Alternatively, the level of expression (e.g., in a cell contacted with a test or candidate compound) can be compared to a suitable control. As used herein, a suitable (or appropriate) control includes an experimental, test, assay, condition, or environment designed or chosen based on the understanding or expectation that no effect or a normal effect (e.g., normal syncytin expression) will be detected. For example, in instances where the test or candidate compound is dissolved or consists of an aqueous or organic solution or solvent, a suitable or appropriate control might include a cell (e.g., a syncytin-expressing cell) contacted with the aqueous or organic solution or solvent, respectively. Alternatively, if the test or candidate compound is in a media extract, media extract absent the test or candidate compound may be an appropriate or suitable control. Moreover, in another exemplary embodiment, a suitable or appropriate control may be a level or value (e.g., a predetermined level or value) determined from a normal or control sample. For example, normal, normalized, or averaged serum syncytin levels (e.g., from a normal pregnant subject or group of such subjects). The candidate compound can then be identified as a modulator of syncytin expression based, for example, on a comparison to a suitable or appropriate control. For example, when expression of syncytin mRNA or protein is greater (statistically significantly greater) in the presence of the candidate compound than in the absence of the compound or in the presence of a suitable control, the candidate compound is identified as a stimulator of syncytin mRNA or protein expression. Alternatively, when expression of syncytin mRNA or protein is less (statistically significantly less) in the presence of the candidate compound than in the absence of the compound or in the presence of a suitable control, the candidate compound is identified as an inhibitor of syncytin mRNA or protein expression. The level of syncytin mRNA or protein expression in the cells (or in a biological sample or isolate thereof) can be determined by methods described herein for detecting syncytin mRNA or protein.

The screening assays of the present invention also include detection of syncytin activity. Detection of syncytin activity can include detection of the cellular localization of the syncytin polypeptide in a cell. For example, syncytin is normally localized to the basal syncytiotrophoblast membrane. Hence, “syncytin activity” is also defined as the normal localization of syncytin to the basal syncytiotrophoblast membrane. Immunohistochemical staining (as in Example 2) can be used to determine the cellular localization of the syncytin polypeptide in a cell.

Syncytin activity can also be determined by a cellular fusion assay, as detailed in Mi et al. (2000) supra. When syncytin is expressed in a cell (e.g., a COS cell or a BeWo cell), it can mediate fusion between that cell and another cell. Therefore, one embodiment of the invention comprises a cell-based assay wherein a cell capable of expressing syncytin is contacted with a test compound and the ability of the test compound to mediate fusion of cells in the assay mixture is assayed. Cell fusion can be monitored by, for example, standard light microscopy, which is known in the art.

In a preferred embodiment, the novel agents identified by the above-described screening assays as syncytin modulators increase the expression or activity of syncytin.

Preferably, in the context of the screening assay methodologies described herein, determining syncytin expression or activity is accomplished in a vessel suitable for reacting assay components (e.g., in a tube, for example, a test tube, in a well, for example, in a microtiter-plate well, on a solid surface, for example, in a droplet or microdroplet, on a chip, or the like). More preferably, determining syncytin expression or activity is accomplished in a high throughput screening assay format or application, optionally assisted by robotics and/or computers for dispensing samples, handling reactants, collecting data, and the like.

This invention further pertains to novel agents identified by the above-described screening assays. Accordingly, it is within the scope of this invention to further use an agent identified as described herein in an appropriate animal model. For example, an agent identified as described herein (e.g., a syncytin modulator) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### II. Methods of Diagnosing Increased Risk for Preeclampsia

As described in Examples 1 and 2, syncytin expression is both decreased and mislocalized within the syncytiotrophoblast cells in preeclampsia. As a result of these abnormalities of syncytin expression and localization, there may be inefficient cell fusion to form the syncytiotrophoblast, leading to the morphologic abnormalities seen in preeclampsia. Lack of efficient cell fusion may cause unstable placental villous structure, and indeed, increased trophoblast deportation into the maternal blood has been reported in pregnancies affected by preeclampsia (Chua, S. et al. (1991) Br. J. Obstet. 98:973-9). Therefore, the level of syncytin in maternal blood will be higher in preeclampsia than in normal pregnancies (i.e., increased trophoblast deportation into the maternal blood in preeclampsia leads to increased syncytin levels in maternal blood).

Accordingly, one embodiment of the present inventions provides a method for diagnosing increased risk for preeclampsia by determining the level of syncytin in a tissue, cell, or biological fluid sample from a pregnant subject. In a preferred embodiment, the level of syncytin in maternal serum or blood is determined. The level of syncytin may also be determined from an amniotic fluid sample or from a tissue sample such as a chorionic villous sample or an endothelial cell sample.

In one embodiment, the invention provides a method for detecting the presence of syncytin in a biological sample. The method involves contacting the biological sample with an agent capable of detecting syncytin protein or nucleic acid molecules (e.g., syncytin mRNA) such that the presence of syncytin is detected in the biological sample. A preferred agent for detecting syncytin mRNA is a labeled or labelable nucleic acid probe capable of hybridizing to syncytin mRNA. The nucleic acid probe can be, for example, the full-length syncytin cDNA of SEQ ID NO: 1, or a portion thereof, such as an oligonucleotide of at least 15, 30, 50, 100, 250 or 500 nucleotides in length and is sufficiently complimentary to specifically hybridize under stringent conditions to syncytin mRNA.

A preferred agent for detecting syncytin protein is a labeled or labelable antibody capable of binding to syncytin protein. Antibodies can be polyclonal, or more preferably, monoclonal. An intact antibody, or a fragment thereof (e.g., Fab or F(ab′)₂) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

As used herein, the term “biological sample” refers to a sample of biological material isolated from a subject, preferably a human subject, or present within a subject, preferably a human subject. The “biological material” can include, for example, tissues, tissue samples, tumors, tumor samples, cells, biological fluids, and purified and/or partially-purified biological molecules. As used herein, the term “isolated”, when used in the context of a biological sample, is intended to indicate that the biological sample has been removed from the subject. In one embodiment, a biological sample comprises a sample which has been isolated from a subject and is subjected to a method of the present invention without further processing or manipulation subsequent to its isolation. In another embodiment, the biological sample can be processed or manipulated subsequent to being isolated and prior to being subjected to a method of the invention. For example, a sample can be refrigerated (e.g., stored at 4° C.), frozen (e.g., stored at −20° C., stored at −135° C., frozen in liquid nitrogen, or cryopreserved using any one of many standard cryopreservation techniques known in the art). Furthermore, a sample can be purified subsequent to isolation from a subject and prior to subjecting it to a method of the present invention. As used herein, the term “purified” when used in the context of a biological sample, is intended to indicate that at least one component of the isolated biological sample has been removed from the biological sample such that fewer components, and consequently, purer components, remain following purification. For example, a serum sample can be separated into one or more components using centrifugation techniques known in the art to obtain partially-purified sample preparation. Furthermore, it is possible to purify a biological sample such that substantially only one component remains. For example, a tissue or tumor sample can be purified such that substantially only the protein or mRNA component of the biological sample remains.

Furthermore, it may be desirable to amplify a component of a biological sample such that detection of the component is facilitated. For example, the mRNA component of a biological sample can be amplified (e.g., by RT-PCR) such that detection of syncytin mRNA is facilitated. As used herein, the term “RT-PCR” (“reverse transcriptase-polymerase chain reaction”) includes subjecting mRNA to the reverse transcriptase enzyme resulting in the production of DNA which is complementary to the base sequences of the mRNA. Large amounts of selected cDNA can then be produced via the polymerase chain reaction which relies on the action of heat-stable DNA polymerase for its amplification action. Alternative amplification methods include, but are not limited to, self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

The detection methods of the present invention can be used to detect syncytin protein or nucleic acid molecules in a biological sample in vitro as well as in vivo. For example, in vitro techniques for detection of syncytin mRNA include Northern hybridizations and in situ hybridizations. In vitro techniques for detection of syncytin protein include enzyme linked immunosorbent assays (ELISAs), Western blots, immunoprecipitations and immunofluorescence.

In order to determine if the level of syncytin in a biological sample from a test subject is abnormal, the level of syncytin from the test subject is compared, for example, to the average level of syncytin determined from women who have had normal pregnancies. If the level of syncytin determined in the test subject is higher (i.e., statistically significantly higher) than the levels for normal pregnancies, the test subject is diagnosed as being at risk for developing preeclampsia.

In one embodiment, a subject having a 1-10% increase in the level of serum syncytin is diagnosed as being at risk for preeclampsia. In another embodiment, a subject having a 10-20% increase in serum syncytin levels is diagnosed as being at risk. In yet another embodiment, a subject having a 20-30%, 30-40%, 40-50%, 50-100%, 100-200%, 200-400% (e.g., 2-fold to 4-fold), 4-fold to 10-fold, 10-fold to 100-fold, 100-fold or greater increased levels, for example, when compared to a suitable or appropriate control, is diagnosed as being at risk for preeclampsia.

In another embodiment, the level of syncytin is determined during the first trimester of pregnancy (i.e., approximately weeks 1-14 of gestation). In a preferred embodiment, the level of syncytin is determined at approximately 14 to 16 weeks gestation, before clinical symptoms typically appear. In a further preferred embodiment, the syncytin levels are determined at least twice, with at least one measurement during weeks 14 to 16 of gestation.

In another embodiment of the invention, in addition to determining the syncytin level in a biological sample from a test subject, levels of other factors may be determined in order to further determine the risk for developing preeclampsia. For example, levels of the cytokine M-CSF are decreased in maternal blood in pregnant women who go on to develop preeclampsia (Keith, J. C. (1996) U.S. Pat. No. 5,580,554; Keith, J. C. (1996) U.S. Pat. No. 5,453,138). Accordingly, one embodiment of the invention provides a method of diagnosing a subject at risk for preeclampsia that includes, in addition to determining the level of syncytin in a biological sample from a test subject, determining the level of M-CSF in the blood, serum, or plasma of a test subject and, optionally, for example, comparing said test subject's syncytin and M-CSF levels to the levels of syncytin and M-CSF in women who have gone on to have normal pregnancies. In general, levels of 500 units/ml M-CSF or above are considered normal. However, M-CSF levels of 500 to about 600 units/ml may be abnormal. A unit of M-CSF is equivalent to approximately 12 pg M-CSF. Thus, units may be measured by direct assay of the amount of M-CSF present (e.g., the exact ng/ml of M-CSF present in the sample), or by bioassay (e.g., the level of activity of the M-CSF in the sample compared to 12 pg of M-CSF). If the test subject's M-CSF level is below about 500 units/ml, optionally, in addition to the subject having a serum syncytin level that is abnormal, the test subject may be diagnosed as being at increased risk for preeclampsia.

In a further embodiment, in addition to determining the level of syncytin in a biological sample from a test subject, the TNFα level in the blood, serum, or plasma of a test subject may also be determined. Serum TNFα levels are decreased, as compared to normal pregnancies, during the early stages of preeclamptic pregnancy. Accordingly, decreased serum TNFα activity levels can be used to further diagnose a test subject as being at risk for preeclampsia. Serum biological activity levels of approximately 40 units/ml or more of TNFα are generally considered normal (Keith et al. (1993) J. Perinatology 13:417-418; Keith et al. (1995) Hypertension and Pregnancy 14:81-90). If a test subject has levels of TNFα that are below normal, optionally, in addition to the subject having a serum syncytin level that is abnormal, the test subject may be diagnosed as being at risk for preeclampsia. In later stages of preeclampsia, however, serum TNFα levels become increased as compared to normal pregnancies.

The present invention also comprises kits for detecting the presence of syncytin in a biological sample. For example, the kit can comprise a labeled or labelable agent capable of detecting syncytin mRNA or polypeptide in a biological sample and a means for determining the amount of syncytin mRNA or polypeptide in a biological sample. The agent can be packaged in a suitable container. The kit can further comprise a means for comparing the amount of syncytin in the sample with a standard (e.g., a chart showing normal and abnormal ranges for syncytin levels or a sample of a suitable or appropriate control) and/or can further comprise instructions for using the kit to detect syncytin mRNA or polypeptide. The kit may also comprise a means for determining the levels of M-CSF and/or TNFα in a biological sample.

### III. Methods of Treatment

In another embodiment, the present invention comprises methods of treatment of preeclampsia and gestational trophoblast disorders. Syncytin expression is decreased in preeclampsia and is also mislocalized within the syncytiotrophoblast cells. Accordingly, the present invention provides methods for treating a subject with or at risk for developing preeclampsia with a syncytin modulator (e.g., peptidomimetic, protein, peptide, nucleic acid, small molecule, or other drug candidate identified by the screening assays described herein; also referred to herein as “active compounds”). In a preferred embodiment, a syncytin modulator increases syncytin expression. In another preferred embodiment, a syncytin modulator increases syncytin activity, as defined herein. A “syncytin modulator”, as used herein, includes any compound that modulates syncytin expression or activity.

In a one embodiment, the amount of a syncytin modulator administered to a subject is a therapeutically effective amount. As used herein, a “therapeutically effective” amount of a compound (e.g., a syncytin modulator) is defined as an amount of a compound sufficient to prevent or treat a disorder or condition (e.g., preeclampsia) that can respond to treatment with said compound. A therapeutically effective amount of a syncytin modulator may also be an amount of a syncytin modulator sufficient to bring syncytin levels (e.g., serum syncytin levels) to normal levels. As used herein, “normal syncytin levels” refers to the average level of syncytin (e.g., the serum level of syncytin polypeptide) measured from a subject or subjects who have gone on to have normal pregnancies. A therapeutically effective amount of a syncytin modulator may also be an amount of a syncytin modulator sufficient to normalize syncytin activity (e.g., correct localization of syncytin to the basal syncytiotrophoblast membrane, as determined by, for example, chorionic villus sampling) in a subject.

The syncytin modulators of the invention can be incorporated into pharmaceutical compositions suitable for administration. Such compositions typically comprise the small molecule, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

In one embodiment, a syncytin modulator is administered beginning late during the first trimester of pregnancy or early during the second trimester. In another embodiment, a syncytin modulator is administered beginning at approximately 14 to 16 weeks gestation or 16 to 18 weeks gestation. In another embodiment, a syncytin modulator is administered beginning at 16 to 18 weeks and continued until syncytin levels in the subject (e.g., serum syncytin levels in the subject) are within normal levels (e.g., syncytin levels determined from subjects with normal pregnancies). Preferably, administration of a syncytin modulator does not continue beyond approximately 30 weeks gestation. Additionally, administration of a syncytin modulator is preferably discontinued before any side effects (e.g., side effects undesirable in a subject) are observed.

In a preferred embodiment, a subject diagnosed as suffering from or at risk for developing preeclampsia may be treated with a syncytin modulator and an additional compound or agent. Such compounds include, but are not limited to, M-CSF and TNFα inhibitors (e.g., etanercept). Serum M-CSF levels are reduced in preeclampsia. Therefore, subjects suffering from or at risk for developing preeclampsia may be treated by administering, in addition to a syncytin modulator, a therapeutically effective amount of M-CSF, as described in Keith, J. C. (1996) U.S. Pat. No. 5,580,554 and Keith, J. C. (1996) U.S. Pat. No. 5,543,138. A therapeutically effective amount of M-CSF may be defined as an amount sufficient to treat a disorder or condition (e.g., preeclampsia) that can respond to M-CSF treatment, or an amount sufficient to bring M-CSF levels (e.g., serum M-CSF levels) to normal levels, as measured in subjects with normal pregnancies.

It has been shown that TNFα production becomes elevated during late pregnancy in preeclampsia (Keith et al. (1993) supra; Keith et al. (1995) supra). Accordingly, it may be desirable to administer a therapeutically effective amount of a TNFα inhibitor (e.g., a polypeptide, for example a soluble TNFα receptor polypeptide or an anti-TNFα antibody, or a small molecule that inhibits TNFα activity) to a subject in conjunction with a syncytin modulator and/or M-CSF. As used herein, a “TNFα inhibitor” includes an agent or compound (e.g., an antibody, polypeptide, nucleic acid molecule, or small molecule) capable of decreasing or reducing the expression or activity of TNFα in a cell or subject, for example, a human subject. Preferably, a TNFα inhibitor inhibits the bioavailability of circulating TNFα in a subject (e.g., decreases the availability of TNFα to interact with a TNFα receptor on a cell (e.g., a cell within a subject). As used herein, a “therapeutically effective amount of a TNFα inhibitor” is defined as an amount sufficient to treat a condition or disorder (e.g., preeclampsia) that can respond to treatment by TNFα inhibition, or an amount sufficient to bring TNFα levels (e.g., serum TNFα levels) to normal levels, as measured in subjects with normal pregnancies.

In a preferred embodiment, the TNFα inhibitor is the compound etanercept (also known by the name Enbrel®, available through Wyeth-Ayerst Pharmaceuticals and Immunex Corporation). Etanercept is a biologically engineered protein that consists of two natural soluble TNF receptor domains linked to the Fc portion of human IgGl (Moreland, L. W. et al. (1997) N. Engl. J. Med. 337:141-147). Etanercept binds to free TNFα and blocks two of the three receptor-binding sites on the TNFα molecule, rendering it inactive. Etanercept binds to TNFα with much greater affinity than the endogenous soluble receptor, making it very effective in inhibiting TNFα interaction with cell-surface receptors (Mohler, K. M. et al. (1993) J. Immunol. 151:1548-1561). Etanercept was originally developed as an agent to block TNFα-mediated inflammatory responses, e.g., rheumatoid arthritis (Fleischmann, R. M. (1999) Clin. Ther. 21:1429-1442; Franklin, C. M. (1999) Sem. Arthritis Rheumatism 29:172-182; Deswal, A. et al. (1999) Circulation 99:3224-3226).

In another embodiment of the invention, a syncytin modulator may be used in the treatment of conditions, other than preeclampsia, that are or may be caused or affected by aberrant syncytin expression or activity. Such conditions include, but are not limited to, gestational trophoblast disorders, e.g., choriocarcinoma, hydatiform mole, placental site tumors, and/or missed or incomplete abortion. Gestational trophoblast disorders consist of disorders or conditions wherein trophoblast cells do not exhibit normal behavior, e.g., growth cessation and/or differentiation into syncytiotrophoblasts. Such disorders can be malignant and cause death in a subject if not treated effectively.

Accordingly, one embodiment of the invention involves treating a subject with a gestational trophoblast disorder with a therapeutically effective amount of a syncytin modulator. A therapeutically effective amount of a syncytin modulator (e.g., a therapeutic amount effective to treat a subject having a gestational trophoblast disorder) includes an amount sufficient to treat a gestational trophoblast disorder, such that the gestational trophoblast disorder is treated or cured. In a preferred embodiment, the syncytin modulator increases the expression or activity of syncytin in the gestational trophoblast disorder.

### IV. Nucleic Acid Molecules and Polypeptides

The sequences of the syncytin nucleic acid molecules and polypeptides used in the methods of the invention are known in the art (Blond, J. L. et al. (1999) J. Virol. 73:1175-85; Mi, S. et al. (2000) Nature 403:785-789; PCT International Publication No. WO 99/60020; and GenBank Accession Nos. AF208161 and NM_{—}014590; the contents of which are incorporated herein by reference). The nucleic acid and polypeptide sequences of syncytin are further set forth as SEQ ID NOs: 1 and 2, respectively. Those skilled in the art will recognize that the syncytin nucleic acid molecules and polypeptides used in the methods of the methods of the invention may include nucleic acid molecules and polypeptides which differ from the sequences set forth in SEQ ID NOs: 1 and 2 but which retain syncytin activity.

For example, the methods of the invention further encompass nucleic acid molecules that differ from the nucleotide sequence shown in SEQ ID NO: 1 due to degeneracy of the genetic code and thus encode the same syncytin protein set forth in SEQ ID NO:2. In another embodiment, the methods of the invention include the use of a nucleic acid molecule having a nucleotide sequence encoding a protein having an amino acid sequence which differs by at least 1, but no greater than 5, 10, 20, 50 or 100 amino acid residues from the amino acid sequence shown in SEQ ID NO:2. The methods of the invention further include the use of a syncytin polypeptide which differs by at least 1, but no greater than 5, 10, 20, 50, 100, or 200 amino acid residues from the amino acid sequence shown in SEQ ID NO:2. If an alignment is needed for this comparison, the sequences should be aligned for maximum homology.

The methods of the invention also include the use of syncytin polypeptides and nucleic acid molecules encoding syncytin polypeptides that contain changes in amino acid residues that are not essential for activity. Such syncytin polypeptides differ in amino acid sequence from SEQ ID NO:2, yet retain biological activity. In one embodiment, the isolated nucleic acid molecule comprises a nucleotide sequence encoding a syncytin polypeptide, wherein the syncytin polypeptide comprises an amino acid sequence at least about 50%, 55%, 60%, 65%, 70%, 75%, 80%, 85%, 88%, 89%, 90%, 95%, 96%, 97%, 98% 99%, 99.1%, 99.2%, 99.3%, 99.4%, 99.5%, 99.6%, 99.7%, 99.8%, 99.9% or more homologous to SEQ ID NO:2, e.g., to the entire length of SEQ ID NO:2.

An isolated nucleic acid molecule encoding a syncytin polypeptide homologous to the protein of SEQ ID NO:2 can be created by introducing one or more nucleotide substitutions, additions or deletions into the nucleotide sequence of SEQ ID NO:1, such that one or more amino acid substitutions, additions or deletions are introduced into the encoded protein. Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a syncytin polypeptide is preferably replaced with another amino acid residue from the same side chain family. Alternatively, in another embodiment, mutations can be introduced randomly along all or part of syncytin polypeptide coding sequence, such as by saturation mutagenesis, and the resultant mutants can be screened for syncytin biological activity to identify mutants that retain activity. Following mutagenesis of SEQ ID NO:1, the encoded protein can be expressed recombinantly and the activity of the protein can be determined.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the syncytin amino acid sequence of SEQ ID NO:2 having 538 amino acid residues, at least 161, preferably at least 215, more preferably at least 269, even more preferably at least 323, and even more preferably at least 377, 430 or 484 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available online through the Genetics Computer Group), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the Genetics Computer Group), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci. 4:11-17 (1988)) which has been incorporated into the ALIGN program (version 2.0 or version 2.0U), using a PAM120 weight residue table, a gap length penalty of 12 and a gap penalty of 4.

This invention is further illustrated by the following examples, which should not be construed as limiting. The contents of all references, patents and published patent applications cited throughout this application, as well as the Sequence Listing, are incorporated herein by reference.

### EXAMPLES

### Example 1

### Comparison of Syncytin Expression in Preeclamptic and Normal Placentae

This example describes the results of an investigation of a potential role for syncytin in preeclampsia. Normal (n=28) and preeclamptic (n=21) placentas were studied by light microscopy, in situ hybridization, and immunohistochemical staining in order to determine whether differences in syncytin gene expression and syncytin protein distribution were present between normal and preeclamptic pregnancy. Previously normotensive pregnancies were diagnosed as preeclamptic if blood pressures were ≧140/90 and there was ≧2+ proteinuria by dipstick determination on two occasions.

Histological Comparison of Placental Sections by Light Microscopy

Placental tissue sections were prepared and processed for routine light microscopic examination. The tissue was stained by hematoxylin and eosin as described by Allen, T. C. (Laboratory Methods in Histotechnology). Placental sections from 30 week and 39 week preeclamptic pregnancies revealed structural abnormalities of syncytiotrophoblast, including increased syncytial knots and proliferation of cytotrophoblasts in the placental villi. In contrast, placental sections from 31 and 40 week, age-matched placentas, obtained from pregnancies without symptoms of preeclampsia, had normal villous syncytiotrophoblast structural features.

Comparison of Syncytin mRNA expression in Placental Sections

In situ hybridization was used to determine and compare the relative syncytin mRNA expression levels between apparently normal and preeclamptic placenta. Placental sections were prepared and processed as described in Mi, S. et al. (2000) supra, and were probed with digoxigenin-labeled syncytin antisense and sense RNA. Sections were stained using alkaline phosphate substrates NBT and BCIP using conditions recommended by the manufacturer. Unlike in normal tissue, there was great difficulty identifying syncytin mRNA in preeclamptic tissues. Compared to normal 40 week placenta, a very weak hybridization signal was detected in the syncytiotrophoblast of the villi from the 32 week preeclamptic placenta.

Comparison of Syncytin mRNA Expression by Northern Blot and RT-PCR

Northern blot hybridization was used to confirm the in situ results set forth above. Northern blots were performed as described in Mi, S. et al. (2000) supra. 2 μg of mRNA from normal or preeclamptic placentas of different gestational ages were heated at 65° C. for 15 minutes in formaldehyde/MOPS agarose gel. The RNA was transferred to a nylon membrane for hybridization to a [³²P]-syncytin probe at 72° C. using Hybridization Express buffer (Clontech Laboratories, Inc., Palo Alto, Calif.). The filter was then washed and autoradiographed using Kodak film.

For apparently normal placentas, a progressive increase in syncytin mRNA expression occurred between 9 to 40 weeks of gestation. However, the syncytin mRNA level from 3 week preeclamptic placenta was lower than apparently normal 9 or 12 week placental levels.

A Taqman RT-PCR kit (PE Biosystems) was used to quantify the syncytin mRNA expression levels. Briefly, syncytin primers

| | |
|---|---|
| 5′-CACAACCAACTCCCAATGCA-3′ | (forward) (SEQ ID NO:3), |
| 5′-AGAGCCATTCAAACAACGATAGG-3′ | (reverse) (SEQ ID NO:4), and |
| 5′-FAM-CTCCTCCCACACAAATAGTCTGCCTACCCT-TAMRA-3′ | (reporter probe, from |
| | Perkin Elmer) (SEQ ID NO:5) |

were used for the quantitation. Human GAPDH control reagents were obtained from Perkin Elmer (#402869 JOE Reporter). The Delta-Delta CT method (Bulletin #2) was used for the calculation of relative levels of RNA expression. The RT-PCR and PCR were performed as described by the manufacturers (MMLV, Cat#Z8025013, Gibco; Taqman™ Gold RT-PCR Kit, Cat#N808-0232, Perkin Elmer).

The results of the quantitative RT-PCR of the syncytin mRNA levels demonstrated that syncytin mRNA levels in the 32 week preeclamptic placenta were about 4-fold lower than syncytin mRNA levels from 21 week placental tissue derived from apparently normal pregnancies, when the mRNA levels were normalized to the housekeeping gene GAPDH.

### Example 2

### Localization of Syncytin Protein in Placental Sections

In order to study the actual distribution of the syncytin protein within the syncytiotrophoblast, immunohistochemical staining of sections of normal and preeclamptic placental tissue was carried out with a specific antibody for syncytin. Placental tissue sections were fixed in 4% paraformaldehyde overnight at 4° C. Immunostaining of placental tissue was performed using a VectastainR ABC-AP kit. A specific Rabbit anti-syncytin antibody described in Mi, S. et al. ((2000) supra) was used to localize syncytin in the syncytiotrophoblast.

In normal placentas, syncytin was predominantly localized to the basal membrane of the syncytiotrophoblast. In contrast, syncytin was localized to the apical membrane of the syncytiotrophoblast in preeclamptic placentas.

To be certain that the syncytiotrophoblast membrane was not inverted, the tissue sections were also stained with endoglin (CD105). Endoglin has been localized previously to the apical membrane of the syncytiotrophoblast (Dagdeviren, A. et al. (1998) Anat. Anz. 180:461-9). Both normal and preeclamptic tissues showed proper localization of endoglin to the apical membrane.

### Example 3

### Measurement of Serum Syncytin Levels as a Diagnostic for Subjects at Risk of Developing or having Preclampsia

Preliminary Western dot blot dilution studies of syncytin in normal versus preeclamptic serum samples demonstrated that syncytin levels are higher in the serum of subjects diagnosed by alternative methodologies as having preeclampsia. Briefly, serum samples from normal subjects (e.g., normal pregnant subjects) and serum samples from preeclamptic subjects were subjected to dilution and resulting diluted samples were analyzed by Western dot blot using a syncytin-specific antibody. Serum samples from preeclamptic women required higher-fold dilution before the syncytin signal by Western dot blot was reduced to baseline as compared to serum samples from normal women.

Accordingly, to diagnose a subject or patient at risk of developing or having preeclampsia, serum levels of syncytin are measured, for example, at 16 weeks in a pregnant subject utilizing standardized methodologies. An exemplary method for measuring syncytin levels in maternal blood is to use an enzyme-linked immunosorbent assay (ELISA). The assay is a triple antibody sandwich using a monoclonal anti-syncytin biotinylated goat anti-rabbit immunoglobulin with streptavidin-peroxidase, and O-phenylenediamine as the chromogen.

The measured syncytin level from the test subject is then compared to an average measured syncytin level from normal pregnant women, and it is determined if there is a (statistically significant) difference (e.g., increase) in serum syncytin levels between the test subject and the average normal subject. If the test subject has a statistically significantly higher level of serum syncytin, the test subject is thereby diagnosed as being at risk for preeclampsia.

### Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating or preventing preeclampsia comprising:
a) contacting a cell with a test compound;
b) determining whether expression or activity of syncytin in the cell is modulated in the presence of the compound; and
c) identifying the compound as being capable of treating or preventing preeclampsia based on the ability of the compound to modulate expression or activity of syncytin.

2. The method of claim 1, wherein the ability of the test compound to modulate syncytin activity in the cell is determined by detecting the ability of the test compound to modulate the cellular localization of syncytin in the cell.

3. The method of claim 1, wherein the ability of the test compound to modulate syncytin activity in the cell is determined by detecting the ability of the test compound to modulate fusion between the cell and a second cell.

4. The method of claim 1, wherein the ability of the compound to modulate syncytin expression is determined by detecting syncytin expression and comparing said expression to a suitable control.

5. The method of claim 1, wherein the cell is selected from the group consisting of a placental cell, a cytotrophoblast, a syncytiotrophoblast, a cell comprising or transfected with a nucleic acid molecule encoding a syncytin polypeptide, and a cell comprising or transfected with a nucleic acid molecule comprising at least one syncytin regulatory element operatively linked to a reporter gene.

6. The method of claim 1, wherein the expression or activity of syncytin is increased.

7. A compound identified according to the method of claim 1.

8. A prognostic method for determining whether a subject is at risk for developing preeclampsia comprising detecting the presence or level of syncytin mRNA or polypeptide in a biological sample obtained from said subject, or isolate of said sample, thereby determining that the subject is at risk for developing preeclampsia based on the presence or level of syncytin detected.

9. A diagnostic method for determining whether a subject has preeclampsia comprising detecting the presence or level of syncytin mRNA or polypeptide in a biological sample obtained from said subject, or isolate of said sample, thereby determining that the subject has preeclampsia.

10. The method of claim 9, further comprising comparing the level of syncytin mRNA or polypeptide in the biological sample obtained from said subject, or isolate of said sample, to the level of syncytin of mRNA or polypeptide in an appropriate control.

11. The method of claim 9, wherein the biological sample is selected from the group consisting of a tissue sample, a tumor sample, an endothelial cell sample, a plasma sample, and a blood sample.

12. The method of claim 9, wherein the biological sample is a serum sample.

13. The method of claim 9, wherein the biological sample is selected from the group consisting of an amniotic fluid sample, a chorionic villus sample, and a placental sample.

14. The method of claim 9, further comprising detecting the presence or level of at least one additional factor in said biological sample.

15. The method of claim 14, wherein said additional factor is selected from the group consisting of TNFα mRNA, TNFα polypeptide, M-CSF mRNA, and M-CSF polypeptide.

16. The method of claim 14, further comprising comparing the level of said additional factor to a suitable control.

17. The method of claim 9, wherein the level of syncytin is determined during the first trimester of pregnancy.

18. The methods of claim 9, wherein the level of syncytin is determined at approximately 14 to 16 weeks of gestation.

19. A method for treating a subject having or at increased risk for preeclampsia comprising administering a therapeutically effective amount of a syncytin modulator, such that said preeclampsia is treated or prevented.

20. The method of claim 19, wherein said therapeutically effective amount of a syncytin modulator comprises an amount sufficient to bring serum syncytin levels to normal serum syncytin levels.

21. The method of claim 19, further comprising administering a therapeutically effective amount of M-CSF.

22. The method of claim 19, further comprising administering a therapeutically effective amount of a TNFα inhibitor.

23. The method of claim 19, further comprising administering a therapeutically effective amount of M-CSF and a therapeutically effective amount of a TNFα inhibitor.

24. The method of 22, wherein the TNFα inhibitor is etanercept.

25. The method of claim 19, wherein said syncytin modulator is administered late during the first trimester or during the second trimester of pregnancy.

26. The methods of claim 19, wherein said syncytin modulator is administered beginning at approximately 14 to 16 weeks gestation and ending at approximately 30 weeks gestation.

27. The methods of claim 19, wherein said syncytin modulator is administered beginning at approximately 14 to 16 weeks gestation and continuing until normal serum syncytin levels are achieved.

28. A method for treating a subject having a gestational trophoblast disorder, said method comprising administering a therapeutically effective amount of a syncytin modulator.

29. The method of claim 28, wherein said gestational trophoblast disorder is selected from the group consisting of a missed or incomplete abortion, choriocarcinoma, hydatiform mole, and placental site tumor.

30. A method for treating a subject having a gestational trophoblast disorder, said method comprising administering a therapeutically effective amount of a syncytin modulator, wherein said syncytin modulator is a compound identified according to the method of claim 1.

31. A kit for use in determining whether a subject has or is at risk for preeclampsia comprising a means for detecting syncytin mRNA or polypeptide in a biological sample or isolate thereof.

32. The kit of claim 31, wherein said means for detecting syncytin mRNA or polypeptide is a labeled or labelable agent capable of detecting syncytin mRNA or polypeptide.

33. The kit of claim 32, wherein the agent is a nucleic acid probe capable of hybridizing to syncytin mRNA.

34. The kit of claim 32, wherein the agent is an antibody capable of specifically binding to syncytin polypeptide.

35. The kit of claim 31, further comprising an appropriate control.

36. The kit of claim 31, further comprising directions for use.
